(19) United States
(12) Patent Application Publication
(10) Pub. No.: US 2003/0118518 A1
(43) Pub. Date: Jun. 26, 2003

(54) Suspension for the treatment of natural hard tissue and method of treatment

(76) Inventors: Rainer Hahn, Tubingen (DE); Dieter Heermann, Hemmingen (DE); Peter Brauner, Affalterbach (DE)

Correspondence Address: King & Jovanovic, PLC, Suite 300, 305 Hoover Blvd., Holland, MI, 49423, US

(21) Appl. No.: 10/282,543

(22) Filed: Oct. 29, 2002

Related U.S. Application Data
(63) Continuation of application No. 09/547,817, filed on Apr. 11, 2000, now abandoned.

(30) Foreign Application Priority Data
Apr. 11, 1999 (DE) 199 16 155.0

(51) Int. Cl. A61K 7/16

(52) U.S. Cl. 424/49

(57) ABSTRACT

A suspension for the ultrasonic preparation of natural hard tissue contains between 5 and 45 wt. % of hard particles, between 2 and 40 wt. % of at least one polyhydric alcohol and between 0.1 and 10 wt. % of dispersants and thickeners The suspension having this composition is capable of being stored and shipped without the risk of the hard particles settling. It constitutes a pumpable concentrate which, during the actual ultrasonic preparation, is diluted with an appropriate amount of water to give the ready-to-use suspension.

The present invention relates to a suspension for the treatment of natural hard tissue, such as dentine, dental enamel, dental calculus, dental cement and dental or bone prostheses, with abrasive hard particles suspended in water or an aqueous solution, in conjunction with the use of vibrating apparatuses or instruments, especially in the ultrasonic range and the treatment of natural hard tissue, such as dentine, dental enamel, dental calculus, dental cement and dental or bone prostheses in conjunction with the use of vibrating apparatuses or instruments, especially in the ultrasonic range, using such a suspension.

The ultrasonic preparation of natural hard tissues has recently become increasingly important. This technique uses tools which are caused to vibrate by means of an ultrasonic vibrator and which are in contact, via a suspension of the type mentioned at the outset, with the hard tissue to be treated. An example of this is described in U.S. Pat. No. 6,139,320 which is incorporated herein by reference. The suspension mentioned in said patent document was unstable insofar as the hard particles were capable of settling over time, so the suspension had to be constantly stirred in an exchangeable reservoir or the hard particles had to be kept in suspension by the passage of a gas. This is very expensive and does not always produce the desired result.

An object of the present invention is to provide a suspension of the type mentioned at the outset which is such that it can be prepared and bottled at the factory, has long-term storage stability and can he shipped, there being no risk of settling of the hard particles and the suspension nevertheless being pumpable at low cost.

A further object of the present invention is to provide a method of treatment using such a suspension.

Accordingly, the invention provides an aqueous suspension concentrate for the treatment of natural hard tissue, such as dentine, dental enamel, dental calculus, dental cement and dental or bone prostheses, with abrasive hard particles suspended in water or an aqueous solution, in conjunction with the use of vibrating apparatuses or instruments, especially in the ultrasonic range,

which suspension contains:

a) between 5 and 45 wt. % of hard particles;

b) between 2 and 40 wt. % of at least one monohydric or polyhydric alcohol or a mixture of monohydric and polyhydric alcohols; and

c) between 0.1 and 10 wt. % of dispersants and thickeners.

The aqueous suspension concentrate according to the invention is an aqueous suspension which, in the desired manner, has long-term storage stability and does not demix. It constitutes a concentrate which is easily diluted with the appropriate amount of water, e.g. in a water to suspension concentrate ratio of 4:1 to 1:1, preferably 3:1 to 1.5:1 and most preferred 2:1:, at the site of the treatment itself, i.e. with the aid of the treatment apparatus or in the handpiece in the patient's oral cavity, before it enters the oral cavity.

The invention further provides a method of treating natural hard tissue and hard tissue prosthesis in conjunction with the use of a vibrating apparatus or instrument with a diluted aqueous suspension, comprising the steps of:

a) forming a diluted suspension of hard particles in an aqueous vehicle by diluting an aqueous suspension concentrate the latter comprising

aa) between 5 and 45 wt. % of hard particles;

bb) between 2 and 40 wt. % of at least one polyhydric alcohol or a mixture of monohydric and polyhydric alcohols; and

cc) between 0.1 and 10 wt. % of dispersants and thickeners,

b) contacting tissue to be treated via the suspension with a tool, and

c) vibrating the tool by means of an ultrasonic vibrator.

In a preferred embodiment of the method the dispersants and thickeners consist of between 0.15 and 3 percent by weight of polysaccharides.

In a further preferred embodiment of the method the dilution of the aqueous suspension concentrate is accomplished with a water to suspension concentrate ratio of 4:1 to 1:1, more preferred 3:1 to 1.5:1 and most preferred 2:1.

The advantageous embodiments of the suspension according to the invention, which are illustrated in greater detail below, are subdivided into three groups: The first group of suspensions is preferably used for smoothing surfaces, removing deposits on hard tissue surfaces or polishing. The second group is particularly suitable for the abrasive treatment or preparation of hard tissue surfaces or of dental or bone prostheses. Finally, the third group is substantially used for the treatment of implant surfaces in the context of the removal of hard or soft coatings.

Starting with the first group:

It is recommended that this group should contain between 5 and 20 wt. %, preferably between 8 and 12 wt. %, of hard particles. Particularly suitable hard particles are calcium phosphates, especially apatites and particularly preferably fluorapatite or hydroxyapatite, silicate ceramics or glasses, and oxide ceramic, e.g. aluminum oxide ceramic particles. The median size of the hard particles is up to a maximum of 100 to 150 μm, preferably approx. 25 μm and particularly preferably about 5 to 10 μm.

The following characteristics have proved particularly satisfactory for the second group of suspensions:

These contain between 5 and 45 wt. %, preferably between 20 and 35 wt. %, of hard particles. Suitable materials are preferably oxide ceramics, e.g. zirconium oxide ceramic particles, and non-oxide ceramics, e.g. boron carbide, boron nitride and silicon nitride. Silicon carbide is very particularly suitable on account of its advantageous green rather than black color. The mean size of the hard particles in this second group is preferably between about 5 μm, preferably between about and 25 about 100 μm and particularly preferably between 40 μm and 50 μm.

Hard particles which are recommended for the third group of suspensions consist of plastic, e.g. polysulfone particles, or plastic precursors, e.g. polysiloxanes or polysilazanes, as well as ceramics prepared by pyrolysis. However, other hard particles suitable for this group are those mentioned above for group 1 or—if the removal of material is desired—those mentioned above for group 2.

A variety of substances are suitable as dispersants and thickeners. For example, the suspension can contain between 2 and 10 wt. %, preferably between 3 and 8 wt. %, of highly disperse silicic acid. Good results are also obtained when the dispersants and thickeners used consist of between 0.1 and 4 wt. %, preferably between 0.3 and 2.5 wt. %, of bentonite.

The dispersants and thickeners preferably consist of between 0.15 and 3 wt. %, particularly preferably of between 0.25 and 1.5 wt. %, of polysaccharide, the latter appropriately being xanthan.

To maintain the sterility of the suspension even over prolonged storage periods, suitable preservatives can be incorporated therein.

It would be particularly favorable per se for the pH of the suspension to be about 11.0. As experiments have shown, the hard particles acquire a negative potential of about −25 mV at this pH. Repulsion of the negative charges on the particle surfaces has a very great stabilizing effect on the suspension. With the present invention, it is also possible to formulate a stable suspension in the pH range between 5 and 9, preferably between 6 and 8. Such a suspension can be applied directly in the oral cavity.

The invention will be described in greater detail below with the aid of two Examples.

### EXAMPLE 1

A suspension with a particularly strong abrasive action has the following composition:

| Chemical name | Content (wt. %) |
|---|---|
| Silicon carbide | 25 |
| Xanthan | 0.55 |
| PHB ester mixture | 0.15 |
| Glycerol | 5 |
| Purified water | ad 100 |

### EXAMPLE 2

Another suspension with a particularly strong abrasive action has the following composition:

| Chemical name | Content (wt. %) |
|---|---|
| Silicon carbide | 30 |
| Xanthan | 0.5 |
| PHB estermixture | 0.2 |
| Glycerol | 12 |
| Phenoxyethanol | 0.5 |
| Ethanol | 3 |
| Purified water | ad 100 |

In Examples 1 and 2 a strongly abrasive action is obtained when the chosen diameter of the silicon carbide particles is in the range of from about 40 to about 50 μm. Particle diameters of from about 5 to about 10 μm result in a more gentle or finishing manner.

### EXAMPLE 3

A less abrasive suspension, suitable especially for polishing purposes, has the following composition:

| Chemical name | Content (wt. %) |
|---|---|
| Hydroxyapatite | 8 |
| Xanthan | 0.55 |
| Glycerol | 15 |
| PHB ester | 0.15 |
| Purified water | ad 100 |

### EXAMPLE 4

Another less abrasive suspension, suitable especially for polishing purposes, has the following composition:

| Chemical name | Content (wt. %) |
|---|---|
| Hydroxyapatite | 8 |
| Xanthan | 0.6 |
| Glycerol | 12 |
| PHB estermixture | 0.2 |
| Phenoxyethanol | 0.5 |
| Ethanol | 3 |
| Purified water | ad 100 |

In the compositions of Examples 3 and 4, the hydroxyapatite can also be replaced with fluorapatite as the hard substance. The result of choosing the particle size in the vicinity of 50 μm or in the range of from about 5 to about 10 μm is forced polishing or, respectively, polishing.

### EXAMPLE 5

Another less abrasive suspension, suitable especially for polishing implants, as the following composition:

| Chemical name | Content (wt. %) |
|---|---|
| Hydroxyapatite | 2.5 |
| Polysiloxan | 5.5 |
| Xanthan | 0.6 |
| Glycerol | 12 |
| PHB ester mixture | 0.2 |
| Phenoxyethanol | 0.5 |
| Ethanol | 3 |
| Purified water | ad 100 |

What is claimed is:

1. A suspension for the treatment of natural hard tissue such as dentine, dental enamel, dental calculus or dental cement, and hard tissue prostheses such as dental or bone prostheses or implants, with abrasive hard particles suspended in water or an aqueous solution, in conjunction with the use of vibrating apparatuses or instruments, especially in the ultrasonic range,
wherein it contains:
a) between 5 and 45 wt. % of hard particles;
b) between 2 and 40 wt. % of an alcohol component comprising at least one monohydric or polyhydric alcohol or a mixture of monohydric or polyhydric alcohols; and
c) between 0.1 and 10 wt. % of dispersants and thickeners.

2. A suspension as claimed in claim 1, wherein it contains between 5 and 20 wt. % of hard particles

3. A suspension as claimed in claim 2, wherein it contains between 8 and 12 wt. % of hard particles.

4. A suspension as claimed in one of claims 1 to 3, wherein the hard particles are calcium phosphates, especially apatites and preferably fluorapatite or hydroxyapatite, silicate ceramics, glasses or oxide ceramic, e.g. aluminum oxide ceramic.

5. A suspension as claimed in one of claims 1 to 4, wherein the maximum mean size of the hard particles is approx. 100 to 150 μm, preferably approx. 25 μm and particularly preferably approx. 5 to 10 μm.

6. A suspension as claimed in claim 1, wherein it contains between 20 and 35 wt. % of hard particles.

7. A suspension as claimed in claim 6, wherein the hard particles are oxide ceramics, e.g. zirconium oxide ceramic, or non-oxide ceramics, e.g. boron carbide, boron nitride or silicon nitride.

8. A suspension as claimed in claim 6, wherein the hard particles are silicon carbide.

9. A suspension as claimed in one of claims 6 to 8, wherein the mean size of the hard particles is between approx. 5 μm and approx. 100 μm, preferably between approx. 25 μm and approx. 100 μm and particularly preferably between 40 μm and 50 μm.

10. A suspension as claimed in one of claims 1 to 9, wherein the hard particles include particles of plastic, e.g. polysulfone particles, or plastic precursors, e.g. polysiloxanes or polysilazanes, or ceramics prepared by pyrolysis, or consist entirely of such particles.

11. A suspension as claimed in one of the preceding claims wherein, the dispersants and thickeners consist of between 2 and 10 wt. % of highly disperse silicic acid.

12. A suspension as claimed in claim 11, wherein it contains between 3 and 8 wt. % of highly disperse silicic acid.

13. A suspension as claimed in one of the preceding claims, wherein the dispersants and thickeners consist of between 0.1 and 4 wt. % of bentonite.

14. A suspension as claimed in claim 13, wherein it contains between 0.3 and 2.5 wt. % of bentonite.

15. A suspension as claimed in one of the preceding claims, wherein the dispersants and thickeners consist of between 0.15 and 3 wt. % of polysaccharides.

16. A suspension as claimed in claim 15, wherein it contains between 0.25 and 1.5 wt. % of polysaccharides.

17. A suspension as claimed in claim 15 or 16, wherein the polysaccharide is xanthan.

18. A suspension as claimed in one of the preceding claims, wherein it contains at least one preservative.

19. A suspension as claimed in one of the preceding claims, wherein the pH is between 5 and 9, preferably between 6 and 8.

20. A suspension according to one of the preceding claims, wherein the alcohol component contains at least one alcohol from the following group: ethanol, propanols, glycerol, propylene glycols and diethylene glycol.

21. A suspension according to one of the preceding claims, wherein it comprises a mixture of inorganic hard particles as claimed in claim 6 or 7 and organic hard particles as claimed in claim 10.
